(19) United States
(12) Patent Application Publication
(10) Pub. No.: US 2004/0265279 A1
(43) Pub. Date: Dec. 30, 2004

(54) Probiotics in the treatment of atypical depression and other disorders characterized by hypothalamic pitiuitary-adrenal axis over-activity

(76) Inventors: Timothy Dinan, Frenches Walk (IE); Liam O'Mahony, Riversdale (IE); Barry Kiely, Simla Villas (IE); Eamonn Quigley, Cappagh (IE); Fergus Shanahan, Fort Cliff (IE); John Kevin Collins, Spur Hill (IE)

Correspondence Address: John J. McDonnell, McDonnell Boehnen Hulbert & Berghoff LLP, 32nd Floor, 300 S. Wacker Drive, Chicago, IL, 60606, US

(21) Appl. No.: 10/841,984

(22) Filed: May 6, 2004

Related U.S. Application Data
(60) Provisional application No. 60/468,812, filed on May 8, 2003.

(51) Int. Cl. A61K 45/00

(52) U.S. Cl. 424/93.4; 424/93.45

(57) ABSTRACT

A method of treating a disorder characterized by hypothalamic-pituitary-adrenal axis over-activity comprising administering to a subject a probiotic bacterium.

### CROSS-REFERENCE TO RELATED APPLICATION

This applications claim the benefit of U.S. Provisional Patent Application Ser. No. 60/468,812, filed May 8, 2003.

### FIELD OF THE INVENTION

The invention is related to the use of probiotics in the treatment of depression and other disorders characterized by hypothalamic-pituitary-adrenal axis over-activity.

### BACKGROUND OF THE INVENTION

Disturbances in hypothalamic-pituitary-adrenal axis (HPA) function are the most consistently demonstrated neuroendocrine abnormalities in major depression. Hypercortisolism and the relative failure to suppress with an overnight dose of dexamethasone are frequently seen. Elevated CSF levels of corticotropin-releasing hormone (CRH)-immunoreactivity, together with blunted CRH-stimulated ACTH release, have also been reported. These changes occur in a setting of adrenal hyperplasia, demonstrable either by CT or MRI imaging (Dinan T Glucocorticoids and the genesis of depressive illness: a psychobiological model. Brit. J. Psychiat. 1984;21 :813-829).

There is also evidence in major depression of significant increases in proinflammatory cytokines such as inteleukin-1 (IL1) and IL6 (Maes M The immunoregulatory effects of antidepressants. Hum. Psychopharmacol. 2001; 16: 95-103.). Both of these cytokines are know to potently activate the HPA and may play a role in sustaining the HPA activation seen in depression. Effective treatment of depression is accompanied by suppression of proinflammatory cytokines and decreased activation of the HPA.

### SUMMARY OF THE INVENTION

A method of treating a disorder characterized by hypothalamic-pituitary-adrenal axis over-activity. The method includes administering to a subject a probiotic bacterium.

### BRIEF DESCRIPTION OF THE DRAWINGS

FIG. 1 illustrates the impact that Bifidobacterium infantis 35624 consumption exerts on serum cytokine levels.

FIG. 2 illustrates the ability of Lb. paracasei AH113 to stimulate IL-10 from human PBMCs.

FIG. 3 demonstrates that lactobacilli and bifidobacteria stimulate IL-10 and TGFβ; while salmonella stimulates proinflammatory cytokines.

### DETAILED DESCRIPTION OF THE INVENTION

The invention provides bacterial strains, which improve immunological control and promote appropriate HPA signalling. A deposit of Lactobacillus salivarius strain UCC 118 was made and accorded the accession number NCIMB 40829. A deposit of Lactobacillus paracasei strain AH113 was made and accorded the accession number NCIMB 41097. A deposit of Bifidobacterium infantis strain 35624 was made at the NCIMB and accorded the accession number NCIMB 41003. Our preliminary data suggests these strains in particular have antidepressant action.

The invention relates to the central effects of probiotic bacteria mediated by modulation of the HPA axis. One of the mechanisms whereby probiotic organisms may impact on the HPA is through interaction with the mucosal epithelium and associated lymphoid structures, thereby causing the host to up-regulate and express molecules, which are anti-inflammatory. These would include cytokines such as IL-10 and TGFβ. The present invention is directed toward probiotic strains, which have immuno-regulatory activity and the capacity to decrease HPA activity.

### EXAMPLE 1

### Probiotic Effects on Serum Cytokine Levels

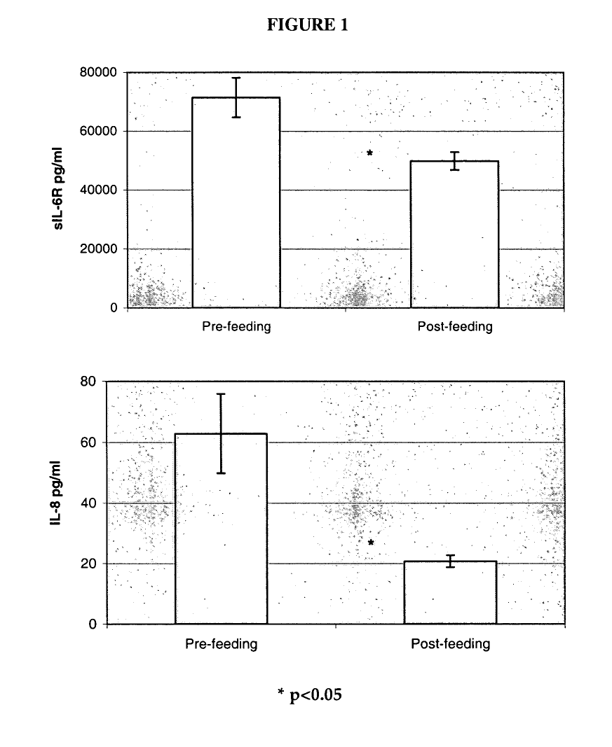

Bifidobacterium infantis 35624 was consumed by 15 healthy volunteers for 3 weeks. Serum cytokine levels were examined pre- and post-consumption. Soluble IL-6 receptor (sIL-6R) and IL-8 levels were significantly decreased following probiotic feeding (FIG. 1). SIL-6R is required for IL-6 signaling while IL-8 is a pro-inflammatory chemokine.

### EXAMPLE 2

### Probiotic Effects on PBMC Cytokine Production

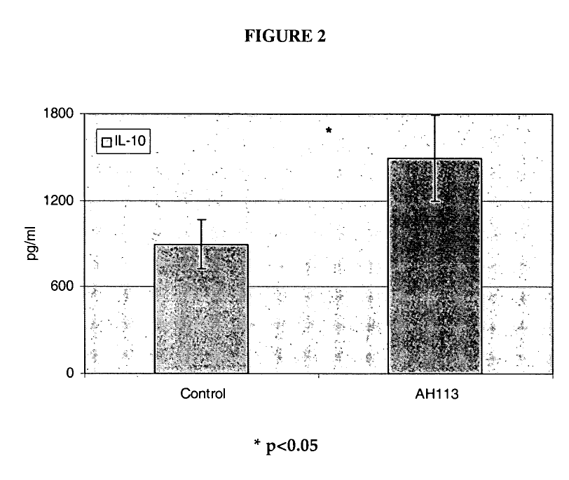

Peripheral blood mononuclear cells were isolated from healthy donors (n=5) by density gradient centrifugation. PBMCs were stimulated with the probiotic bacterial strain for a 72 hour period at 37° C. At this time culture supernatants were collected, centrifuged, aliquoted and stored at −70° C. until being assessed for IL-10 levels using ELISAs (R&D Systems). Lb. paracasei AH113 stimulated IL-10 secretion from PBMCs (FIG. 2).

### EXAMPLE 3

### Probiotic Effects on Lymph Node Cytokine Production

Human mesenteric lymph node cells (MLNCs) were isolated from resected specimens and stimulated in vitro with Lb. salivarius UCC118, B. infantis 35624 or Salmonella typhimurium for 3 days. Supernatants were harvested and cytokine levels assessed using ELISAs.

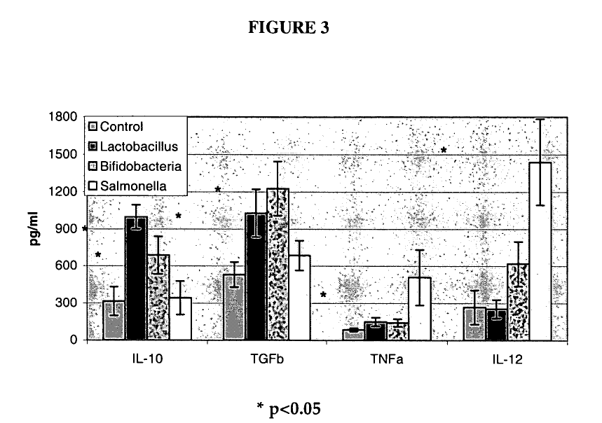

The probiotic bacteria Lb. salivarius UCC118 and B. infantis 35624 stimulated IL-10 and TGFβ production, with no induction of proinflammatory cytokines such as TNFα or IL-12. In contrast, co-incubation with S. typhimurium induced proinflammatory cytokine production with no IL-10 or TGFβ release. Production of IL-10 or TGFβ in vivo following contact with these probiotic bacteria could down-regulate aberrant HPA activity (FIG. 3).

### EXAMPLE 4

### Case Reports

Patient 1 was a 36 year old man with a 6 week history of depression. He had previously been depressed 4 years ago. He complained of low mood, anhedonia, early morning wakening, anorexia, loss of 2 kg weight, significant anxiety and negative thoughts about the future. He had a Hamilton depression score of 23.

He had a strong family history of mood disorder. He was medication free at the time of presentation. He was given bifidobacteria in a milk suspension which he took daily for 12 weeks. At the end of week 2 there was a significant improvement in his mental state and by week 3 he was free of symptoms with a Hamilton score of 4.

Patient 2 was a 56 year old woman with a 2 year history of fatigue. All physical investigations were negative and on the basis of her symptoms she was diagnosed using Center for Disease Control criteria as suffering from chronic fatigue syndrome. She failed to respond to a course of cognitive behaviour therapy and a course of a selective serotonin reuptake inhibitor. She was given bifidobacteria in a milk suspension which she took daily for 20 weeks.

By week 6 she noted an improvement in her energy levels. Six weeks later she was walking 2 miles each day. Such exertion would have been impossible on initial presentation. By week 18 she begun working part-time.

Although various specific embodiments of the present invention have been described herein, it is to be understood that the invention is not limited to those precise embodiments and that various changes or modifications can be affected therein by one skilled in the art without departing from the scope and spirit of the invention.

What is claimed is:

1. A method of treating depression comprising administering to a subject a bacterial strain.

2. The method of claim 1 wherein the bacterial strain is a Lactobacillus strain.

3. The method of claim 1 wherein the bacterial strain is a Bifidobacterium strain.

4. The method of claim 1 wherein the bacterial strain is a probiotic bacterium.

5. The method of claim 4 wherein the probiotic bacterium is Lactobacillus salivarius.

6. The method of claim 4 wherein the probiotic bacterium is Lactobacillus casei.

7. The method of claim 4 wherein the probiotic bacterium is Bifidobacterium longum/infantis.

8. The method of claim 1 wherein the bacterial strain is Lactobacillus salivarius strain UCC 118.

9. The method of claim 1 wherein the bacterial strain is Lactobacillus casei strain AH113.

10. The method of claim 1 wherein the bacterial strain is Bifidobacterium infantis strain 35624.

11. A method of treating a disorder characterized by hypothalamic-pituitary-adrenal axis over-activity comprising administering to a subject a probiotic bacterium.

12. The method of claim 11 wherein the disorder is selected from the group consisting of Cushing's disease, anxiety, psychosocial disorders, stress, atypical depression, panic disorders and fatigue.

13. The method of claim 11 wherein the probiotic bacterium is selected from the group consisting of a Lactobacillus strain, Bifidobacterium strain, Lactobacillus salivarius, Lactobacillus casei, Bifidobacterium longum/infantis, Lactobacillus salivarius strain UCC 118, Lactobacillus casei strain AH113, and Bifidobacterium infantis strain 35624.

14. The method of claim 11 where the subject is human, canine, feline, bovine, equine, mammalian or reptilian.

15. A bacterial cell from the group selected from Lactobacillus strain, Bifidobacterium strain, Lactobacillus salivarius, Lactobacillus casei, Bifidobacterium longum/infantis, Lactobacillus salivarius strain UCC 118, Lactobacillus casei strain AH113, and Bifidobacterium infantis strain 35624, wherein the bacteria are dead, or components or mutants thereof.

16. A pharmaceutical composition comprising a probiotic, or an active derivative, fragment or mutant thereof, for administration in a food substance, a tablet, capsule or other formulation.

17. A pharmaceutical composition comprising a probiotic, or an active derivative, fragment or mutant thereof, for enteral or parenteral administration.
